(19) United States
(12) Patent Application Publication
(10) Pub. No.: US 2024/0324909 A1
(43) Pub. Date: Oct. 3, 2024

(54) HEARING TESTS FOR AUDITORY DEVICES

(71) Applicant: Sony Group Corporation, Tokyo (JP)

(72) Inventors: James R. Milne, Romona, CA (US); Gregory Carlsson, Santee, CA (US); Justin Kenefick, San Diego, CA (US); Allison Burgueno, Oceanside, CA (US)

(73) Assignee: Sony Group Corporation, Tokyo (JP)

(21) Appl. No.: 18/128,689

(22) Filed: Mar. 30, 2023

(51) Int. Cl. A61B 5/12

(52) U.S. Cl. CPC A61B 5/123

(57) ABSTRACT

A computer-implemented method performed on a user device includes receiving a signal from an auditory device. The method further includes determining whether a user wants to take a hearing test. The method further includes implementing threshold-level testing. The method further includes implementing frequency gain balance testing. The method further includes implementing speech-clarity testing. The method further includes generating a hearing profile based on one or more selected from the group of the threshold-level testing, the frequency gain balance testing, the speech-clarity testing, and combinations thereof.

### BACKGROUND

On Oct. 22, 2022, the Food and Drug Administration's ruling went into effect that allows consumers to purchase over-the-counter hearing aids without a medical exam, prescription, or professional fitting. Currently most hearing tests involve listening to test tones that are based on a single frequency at a time, such as 1 kilohertz and then the amplitude is increased until the listener can hear that tone. This method fails to simulate how humans hear in the real world. For example, individual sounds contain complex harmonics. Furthermore, humans detect multiple sounds in an environment at any given time, such as when music is playing.

### SUMMARY

In some embodiments, a computer-implemented method performed on a user device includes receiving a signal from an auditory device. The method further includes determining whether a user selected to take a hearing test. The method further includes implementing threshold-level testing. The method further includes implementing frequency gain balance testing. The method further includes implementing speech-clarity testing. The method further includes generating a hearing profile based on at least one selected from the group of the threshold-level testing, the frequency gain balance testing, the speech-clarity testing, and combinations thereof.

In some embodiments, the method further includes responsive to the user declining to take the hearing test, applying a default profile. In some embodiments, implementing the threshold-level testing includes: instructing the auditory device to play a test sound at a listening band, determining whether a confirmation was received that the user heard the test sound, responsive to not receiving the confirmation, instructing the auditory device to increase a decibel level of the test sound until the confirmation is received or the test sound is played at a decibel level that meets a decibel threshold, responsive to receiving the confirmation that the user heard the test sound or the test sound was played at the decibel threshold, advancing the listening band to a subsequent increment, and continuing to repeat previous steps until the listening band meets a total listening band. In some embodiments, the method further includes generating a user interface with an option for the user to select a number of listening bands. In some embodiments, the threshold-level testing includes playing background noise with the test sound, where the background noise is at least one selected from the group of white noise, voices, music, and combinations thereof.

In some embodiments, implementing the frequency gain balance testing includes: instructing the auditory device to play a first test sound at listening band N and a second test sound at listening band N+1, where the first test sound is a reference test sound, determining whether a confirmation was received that the first test sound and the second test sound were perceived to be a same volume, responsive to not receiving the confirmation, raising a decibel level of the second test sound until the first test sound and the second test sound are perceived to be the same volume, responsive to receiving the confirmation, advancing the listening band N so that N=N+1, where the second test sound becomes the reference test sound and is compared to a subsequent test sound at listening band N+1, continuing to repeat the previous steps until the listening band N meets a total listening band, and updating the hearing profile to include the frequency gain balance testing. In some embodiments, the first test sound is played at a decibel level at which conversations are held and the second test sound is played at a threshold of hearing for a corresponding listening band as determined during the threshold-level testing. In some embodiments, the frequency gain balance testing includes repeating the previous steps while playing background noise with the first test sound and the second test sound, where the background noise is at least one selected from the group of white noise, voices, music, and combinations thereof.

In some embodiments, implementing the speech-clarity testing includes: instructing the auditory device to play a speaking test, determining whether a confirmation was received that the user is satisfied with the speaking test, responsive to not receiving the confirmation that the user is satisfied with the speaking test, modifying the speaking test, continuing to repeat the previous steps until the user is satisfied with the speaking test, determining whether the user wants to repeat the speaking test with a voice of a different gender, and responsive to completing the previous steps with the voice of a different gender or the user not wanting to repeat the speaking test with the voice of a different gender, updating the hearing profile. In some embodiments, implementing the speech-clarity testing further includes playing the speaking test with one or more background noises until the one or more background noises are played. In some embodiments, the threshold-level testing, the frequency gain balance testing, and the speech-clarity testing are implemented on a first ear and then on a second ear and the hearing profile includes different profiles for the first ear and the second ear. In some embodiments, the auditory device is a hearing aid, earbuds, headphones, or a speaker device. In some embodiments, the method further includes determining one or more presets that correspond to user preferences and transmitting the hearing profile and the one or more presets to the auditory device.

In some embodiments, an apparatus includes one or more processors and logic encoded in one or more non-transitory media for execution by the one or more processors and when executed are operable to: receive a signal from an auditory device, determine whether a user selected to take a hearing test, implement threshold-level testing, implement frequency gain balance testing, implement speech-clarity testing, and generate a hearing profile based on at least one selected from the group of the threshold-level testing, the frequency gain balance testing, the speech-clarity testing, and combinations thereof.

In some embodiments, implementing the threshold-level testing includes: instructing the auditory device to play a test sound at a listening band, determining whether a confirmation was received that the user heard the test sound, responsive to not receiving the confirmation, instructing the auditory device to increase a decibel level of the test sound until the confirmation is received or the test sound is played at a decibel level that meets a decibel threshold, responsive to receiving the confirmation that the user heard the test sound or the test sound was played at the decibel threshold, advancing the listening band to a subsequent increment, and continuing to repeat previous steps until the listening band meets a total listening band.

In some embodiments, implementing the frequency gain balance testing includes: instructing the auditory device to play a first test sound at listening band N and a second test sound at listening band N+1, where the first test sound is a reference test sound, determining whether a confirmation was received that the first test sound and the second test sound were perceived to be a same volume, responsive to not receiving the confirmation, raising a decibel level of the second test sound until the first test sound and the second test sound are perceived to be the same volume, responsive to receiving the confirmation, advancing the listening band N so that N=N+1, where the second test sound becomes the reference test sound and is compared to a subsequent test sound at listening band N+1, continuing to repeat the previous steps until the listening band N meets a total listening band, and updating the hearing profile to include the frequency gain balance testing.

In some embodiments, software is encoded in one or more computer-readable media for execution by the one or more processors and when executed is operable to: receive a signal from an auditory device, determine whether a user selected to take a hearing test, implement threshold-level testing, implement frequency gain balance testing, implement speech-clarity testing, and generate a hearing profile based on at least one from the group of the threshold-level testing, the frequency gain balance testing, the speech-clarity testing, and combinations thereof.

In some embodiments, implementing the threshold-level testing includes: instructing the auditory device to play a test sound at a listening band, determining whether a confirmation was received that the user heard the test sound, responsive to not receiving the confirmation, instructing the auditory device to increase a decibel level of the test sound until the confirmation is received or the test sound is played at a decibel level that meets a decibel threshold, responsive to receiving the confirmation that the user heard the test sound or the test sound was played at the decibel threshold, advancing the listening band to a subsequent increment, and continuing to repeat previous steps until the listening band meets a total listening band.

In some embodiments, implementing the frequency gain balance testing includes: instructing the auditory device to play a first test sound at listening band N and a second test sound at listening band N+1, where the first test sound is a reference test sound, determining whether a confirmation was received that the first test sound and the second test sound were perceived to be a same volume, responsive to not receiving the confirmation, raising a decibel level of the second test sound until the first test sound and the second test sound are perceived to be the same volume, responsive to receiving the confirmation, advancing the listening band N so that N=N+1, where the second test sound becomes the reference test sound and is compared to a subsequent test sound at listening band N+1, continuing to repeat the previous steps until the listening band N meets a total listening band, and updating the hearing profile to include the frequency gain balance testing.

In some embodiments, implementing the speech-clarity testing includes: instructing the auditory device to play a speaking test, determining whether a confirmation was received that the user is satisfied with the speaking test, responsive to not receiving the confirmation that the user is satisfied with the speaking test, modifying the speaking test, continuing to repeat the previous steps until the user is satisfied with the speaking test, determining whether the user wants to repeat the speaking test with a voice of a different gender, and responsive to completing the previous steps with the voice of a different gender or the user not wanting to repeat the speaking test with the voice of a different gender, updating the hearing profile.

The technology advantageously creates a more realistic hearing profile that identifies certain hearing conditions that are missed by traditional hearing profiles.

A further understanding of the nature and the advantages of particular embodiments disclosed herein may be realized by reference of the remaining portions of the specification and the attached drawings.

### BRIEF DESCRIPTION OF THE DRAWINGS

FIG. 1 is a block diagram of an example network environment according to some embodiments described herein.

FIG. 2 is an illustration of example auditory devices according to some embodiments described herein.

FIG. 3 is a block diagram of an example computing device according to some embodiments described herein.

FIG. 4A is an example user interface for specifying a type of auditory device, according to some embodiments described herein.

FIG. 4B is an example user interface for selecting a level of granularity of the hearing test according to some embodiments described herein.

FIG. 4C is an example user interface for frequency gain balance testing according to some embodiments described herein.

FIG. 4D illustrates an example user interface for speech-clarity testing according to some embodiments described herein.

FIG. 5 is an illustration of an example audiogram of a right ear and a left ear according to some embodiments described herein.

FIG. 6 illustrates a flowchart of a method to implement a hearing test according to some embodiments described herein.

FIG. 7 illustrates a flowchart of a method to implement threshold-level testing according to some embodiments described herein.

FIG. 8 illustrates a flowchart of a method to implement frequency gain balance for music according to some embodiments described herein.

FIG. 9 illustrates a flowchart of a method to implement speech clarity according to some embodiments described herein.

### DETAILED DESCRIPTION OF EMBODIMENTS

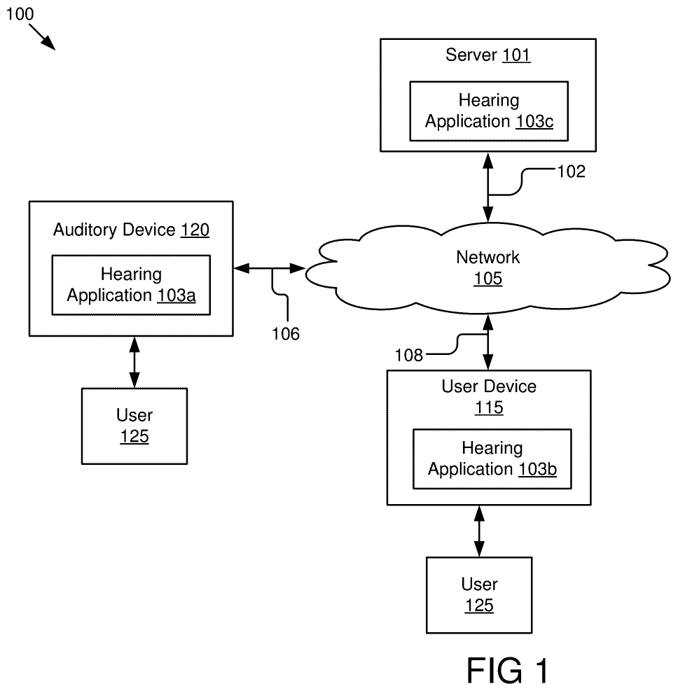

FIG. 1 illustrates a block diagram of an example environment 100. In some embodiments, the environment 100 includes an auditory device 120, a user device 115, and a server 101. A user 125 may be associated with the user device 115 and/or the auditory device 120. In some embodiments, the environment 100 may include other servers or devices not shown in FIG. 1. In FIG. 1 and the remaining figures, a letter after a reference number, e.g., “103a,” represents a reference to the element having that particular reference number (e.g., a hearing application 103a stored on the user device 115). A reference number in the text without a following letter, e.g., “103,” represents a general reference to embodiments of the element bearing that reference number (e.g., any hearing application).

The auditory device 120 may include a processor, a memory, a speaker, and network communication hardware. The auditory device 120 may be a hearing aid, earbuds, headphones, or a speaker device. The speaker device may include a standalone speaker, such as a soundbar or a speaker that is part of a device, such as a speaker in a laptop, tablet, phone, etc.

The auditory device 120 is communicatively coupled to the network 105 via signal line 106. Signal line 106 may be a wired connection, such as Ethernet, coaxial cable, fiber-optic cable, etc., or a wireless connection, such as Wi-Fi®, Bluetooth®, soundwaves, or other wireless technology.

In some embodiments, the auditory device 120 includes a hearing application 103a that performs hearing tests. For example, the user 125 may be asked to identify sounds emitted by speakers of the auditory device 120 and the user may provide user input, for example, by pressing a button on the auditory device 120, such as when the auditory device is a hearing aid, earbuds, or headphones. In some embodiments where the auditory device 120 is larger, such as when the auditory device 120 is a speaker device, the auditory device 120 may include a display screen that receives touch input from the user 125.

In some embodiments, the auditory device 120 communicates with a hearing application 103b stored on the user device 115. During testing, the auditory device 120 receives instructions from the user device 115 to emit test sounds at particular decibel levels. Once testing is complete, the auditory device 120 receives a hearing profile that includes instructions for how to modify sound based on different factors, such as frequencies, types of sounds, etc. The auditory device 120 may also receive instructions from the user device 115 to emit different combinations of sounds in relation to determining user preferences that are memorialized as one or more presets. For example, the auditory device 120 may identify an environment, such as a crowded room, where multiple people are speaking and modify the sound based on one or more presets. The auditory device 120 may amplify certain sounds and filter out other sounds based on the hearing profile and the one or more presets that convert the modified sounds to sound waves that are output through a speaker associated with the auditory device 120.

The user device 115 may be a computing device that includes a memory, a hardware processor, and a hearing application 103b. The user device 115 may include a mobile device, a tablet computer, a laptop, a desktop computer, a mobile telephone, a wearable device, a head-mounted display, a mobile email device, or another electronic device capable of accessing a network 105 to communicate with one or more of the server 101 and the auditory device 120.

In the illustrated implementation, user device 115 is coupled to the network 105 via signal line 108. Signal line 108 may be a wired connection, such as Ethernet, coaxial cable, fiber-optic cable, etc., or a wireless connection, such as Wi-Fi®, Bluetooth®, soundwaves, or other wireless technology. The user device 115 is used by way of example. While FIG. 1 illustrates one user device 115, the disclosure applies to a system architecture having one or more user devices 115.

In some embodiments, the hearing application 103b includes code and routines operable to connect with the auditory device 120 to receive a signal, such as by making a connection via Bluetooth® or Wi-Fi®; determine whether a user selected to take a hearing test; implement threshold-level testing; implement frequency gain balance testing; implement speech-clarity testing; generate a hearing profile based on one or more selected from the group of the threshold-level testing, the frequency gain balance testing, the speech-clarity testing, and combinations thereof; and transmit the hearing profile to the auditory device 120.

In some embodiments where the user declines to take a hearing test, the hearing application 103b transmits a default hearing profile to the auditory device 120 or instructs the auditory device 120 to implement a default hearing profile. The default hearing profile may be further divided based on demographic information, such as a profile based on sex, age, known hearing conditions, etc.

The server 101 may include a processor, a memory, and network communication hardware. In some embodiments, the server 101 is a hardware server. The server 101 is communicatively coupled to the network 105 via signal line 102. Signal line 102 may be a wired connection, such as Ethernet, coaxial cable, fiber-optic cable, etc., or a wireless connection, such as Wi-Fi®, Bluetooth®, or other wireless technology. In some embodiments, the server includes a hearing application 103c. In some embodiments and with user consent, the hearing application 103c on the server 101 maintains a copy of the hearing profile and the one or more presets. In some embodiments, the server 101 maintains audiometric profiles generated by an audiologist for different situations, such as an audiometric profile of a person with no hearing loss, an audiometric profile of a man with mild hearing loss, an audiometric profile of a woman with severe hearing loss, etc.

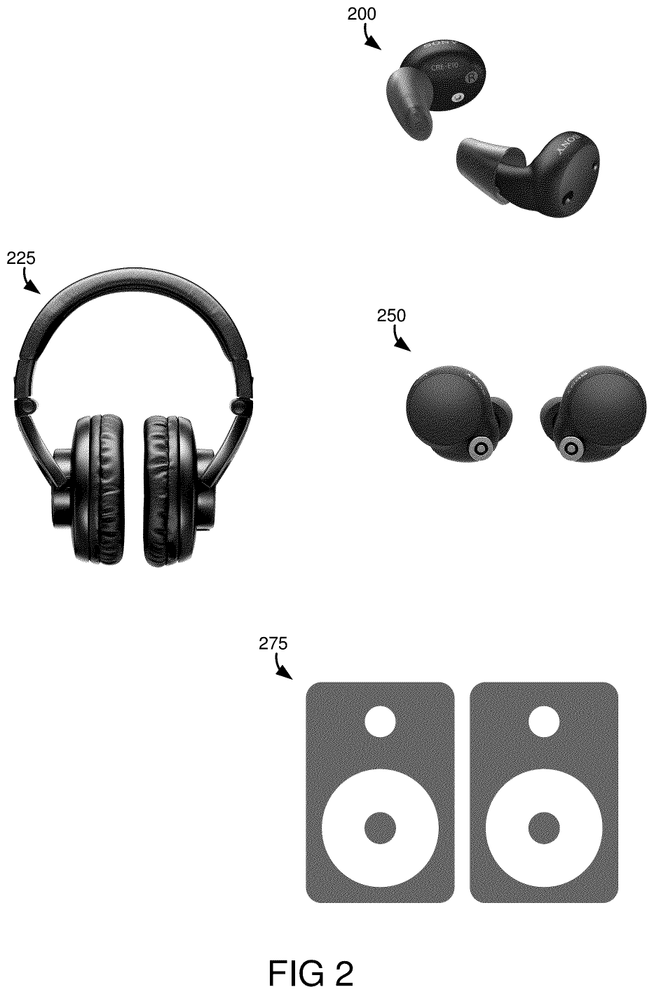

FIG. 2 illustrates example auditory devices. Specifically, FIG. 2 illustrates a hearing aid 200, headphones 225, earbuds 250, and a speaker device 275. In some embodiments, each of the auditory devices is operable to receive instructions from the hearing application 103 to produce sounds that are used to test a user's hearing and modify sounds produced by the auditory device based on a hearing profile. The auditory devices may be Sony products or other products.

### Example Computing Device 300

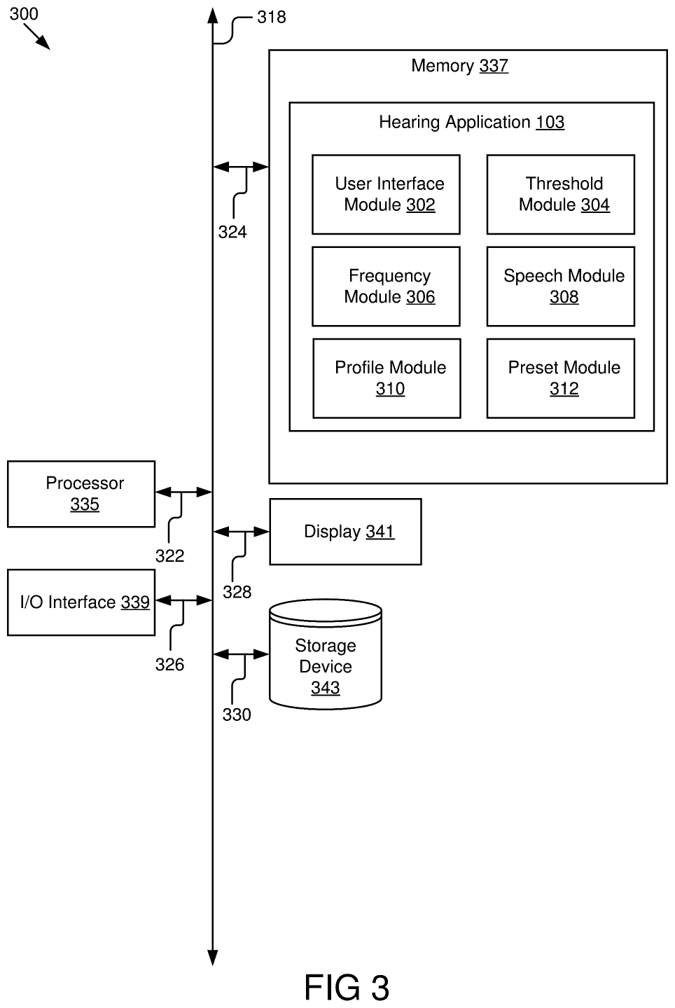

FIG. 3 is a block diagram of an example computing device 300 that may be used to implement one or more features described herein. The computing device 300 can be any suitable computer system, server, or other electronic or hardware device. In one example, the computing device 300 is the user device 115 illustrated in FIG. 1.

In some embodiments, computing device 300 includes a processor 335, a memory 337, an Input/Output (I/O) interface 339, a display 341, and a storage device 343. The processor 335 may be coupled to a bus 318 via signal line 322, the memory 337 may be coupled to the bus 318 via signal line 324, the I/O interface 339 may be coupled to the bus 318 via signal line 326, the display 341 may be coupled to the bus 318 via signal line 328, and the storage device 343 may be coupled to the bus 318 via signal line 330.

The processor 335 can be one or more processors and/or processing circuits to execute program code and control basic operations of the computing device 300. A processor includes any suitable hardware system, mechanism or component that processes data, signals or other information. A processor may include a system with a general-purpose central processing unit (CPU) with one or more cores (e.g., in a single-core, dual-core, or multi-core configuration), multiple processing units (e.g., in a multiprocessor configuration), a graphics processing unit (GPU), a field-programmable gate array (FPGA), an application-specific integrated circuit (ASIC), a complex programmable logic device (CPLD), dedicated circuitry for achieving functionality, or other systems. A computer may be any processor in communication with a memory.

The memory 337 is typically provided in computing device 300 for access by the processor 335 and may be any suitable processor-readable storage medium, such as random access memory (RAM), read-only memory (ROM), Electrical Erasable Read-only Memory (EEPROM), Flash memory, etc., suitable for storing instructions for execution by the processor or sets of processors, and located separate from processor 335 and/or integrated therewith. Memory 337 can store software operating on the computing device 300 by the processor 335, including the hearing application 103.

The I/O interface 339 can provide functions to enable interfacing the computing device 300 with other systems and devices. Interfaced devices can be included as part of the computing device 300 or can be separate and communicate with the computing device 300. For example, network communication devices, storage devices (e.g., the memory 337 or the storage device 343), and input/output devices can communicate via I/O interface 339. In some embodiments, the I/O interface 339 can connect to interface devices such as input devices (keyboard, pointing device, touchscreen, microphone, sensors, etc.) and/or output devices (display 341, speakers, etc.).

The display 341 may connect to the I/O interface 339 to display content, e.g., a user interface, and to receive touch (or gesture) input from a user. The display 341 can include any suitable display device such as a liquid crystal display (LCD), light emitting diode (LED), or plasma display screen, cathode ray tube (CRT), television, monitor, touchscreen, or other visual display device.

The storage device 343 stores data related to the hearing application 103. For example, the storage device 343 may store hearing profiles generated by the hearing application 103, sets of test sounds for testing speech, sets of test sounds for testing music, etc.

Although particular components of the computing device 300 are illustrated, other components may be added or removed.

### Example Hearing Application 103

In some embodiments, the hearing application 103 includes a user interface module 302, a threshold module 304, a frequency module 306, a speech module 308, a profile module 310, and a preset module 312.

The user interface module 302 generates a user interface. In some embodiments, the user interface module 302 includes a set of instructions executable by the processor 335 to generate the user interface. In some embodiments, the user interface module 302 is stored in the memory 337 of the computing device 300 and can be accessible and executable by the processor 335.

In some embodiments, a user downloads the hearing application 103 onto a computing device 300. The user interface module 302 may generate graphical data for displaying a user interface where the user provides input that the profile module 310 uses to generate a hearing profile for a user. For example, the user may provide a username and password, input their name, and provide an identification of an auditory device (e.g., identify whether the auditory device is a hearing aid, headphones, earbuds, or a speaker device).

In some embodiments, the user interface includes an option for specifying a particular type of auditory device and a particular model that is used during testing. For example, the hearing aids may be Sony C10 self-fitting over-the-counter hearing aids (model CRE-C10) or E10 self-fitting over-the-counter hearing aids (model CRE-E10). The identification of the type of auditory device is used for, among other things, determining a beginning decibel level for the test sounds. For example, because hearing aids, earbuds, and headphones are so close to the ear (and are possibly positioned inside the ear), the beginning decibel level for a hearing aid is 0 decibels. For testing of a speaker device, the speaker device should be placed a certain distance from the user and the beginning decibel level may be modified according to that distance. For example, for a speaker device that is within five inches of the user, the beginning decibel level may be 10 decibels.

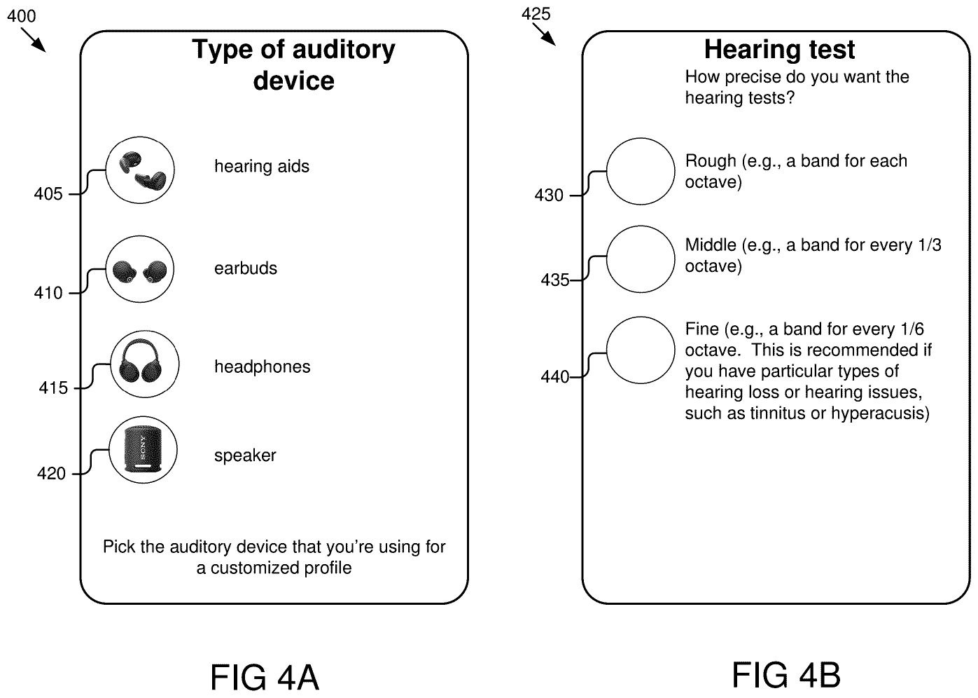

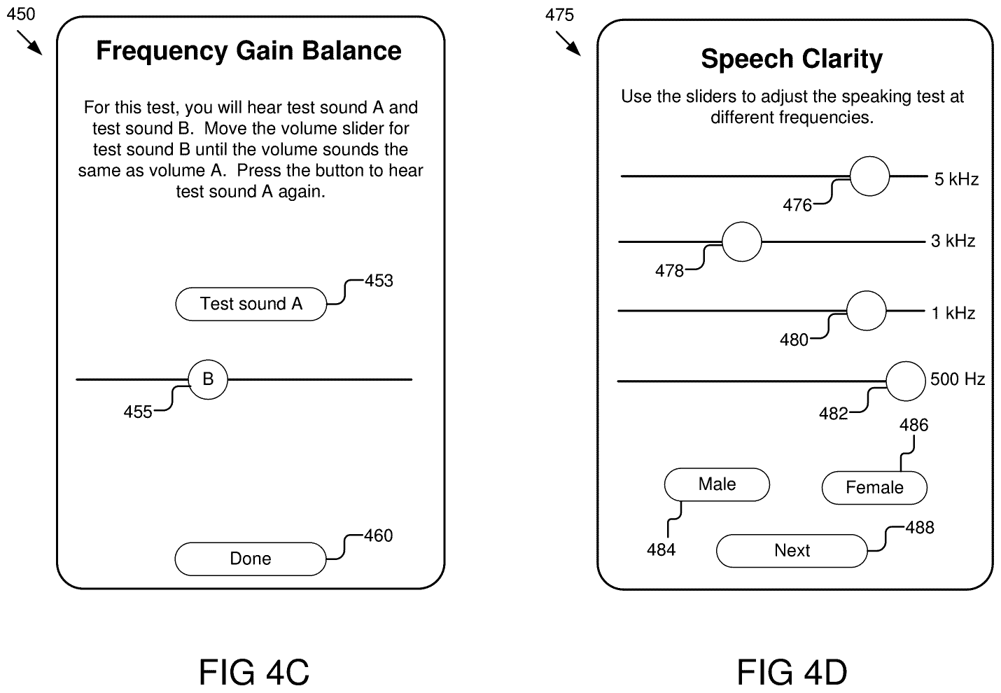

Turning to FIG. 4A, an example user interface 400 for specifying a type of auditory device is illustrated. The user interface module 302 generates graphical data for displaying a list of types of auditory devices. In this example, the user may select the type of auditory device by selecting the hearing aids icon 405 for hearing aids, the earbuds icon 410 for earbuds, the headphones icon 415 for headphones, or the speaker icon 420 for a speaker.

In some embodiments, once the user selects a type of auditory device, the user interface module 302 may generate graphical data to display more types of audio devices, manufacturers, and/or and models for the type of auditory device. For example, if a user selects the headphones icon 415, the user interface module 302 may display an option between wired and wireless headphones. Once the user selects between wired and wireless headphones, the user interface module 302 may display a list of manufacturers.

Once the user selects a particular manufacturer, the user interface module 302 may display different models offered by the manufacturers. For example, if the user selects Sony wireless headphones, the user interface module 302 may generate graphical data for displaying a list of models of wireless Sony headphones. For example, the list may include WH-1000XM4 wireless Sony headphones and WH-CH710N wireless Sony headphones. Other Sony headphones may be selected.

The user interface module 302 may generate graphical data for displaying a user interface that enables a user to make a connection between the computing device 300 and the auditory device. For example, the auditory device may be Bluetooth enabled and the user interface module 302 may generate graphical data for instructing the user to put the auditory device in pairing mode. The computing device 300 may receive a signal from the auditory device via the I/O interface 339 and the user interface module 302 may generate graphical data for displaying a user interface that guides the user to select the auditory device from a list of available devices.

The user interface module 302 generates graphical data for displaying a user interface that allows a user to select a hearing test or decline to take a hearing test. For example, the user interface may include a button for selecting a particular hearing test, a link for skipping the hearing test, etc. If the profile module 310 determines that the user declines to take a hearing test, the profile module 310 may apply a default profile. In some embodiments, the user interface provides an option to select one or more of threshold-level testing, frequency gain balance testing, and speech-clarity testing. In some embodiments, the user may select which type of test is performed first. In some embodiments, the user interface first presents threshold-level testing, then frequency gain testing, and then speech-clarity testing. In some embodiments, before testing begins the user interface includes an instruction for the user to move to an indoor area that is quiet and relatively free of background noise.

In some embodiments, the user interface includes an option for specifying if a user has one or more auditory conditions, such as tinnitus, hyperacusis, or phonophobia. If the user has a particular condition, the corresponding modules may modify the hearing tests accordingly. For example, hyperacusis is a condition where a user experiences discomfort from very low intensity sounds and less discomfort as the frequency increases. As a result, if a user identifies that they have hyperacusis, the threshold module 304 may instruct the auditory device to emit sounds at an initial lower decibel level that is 20-25 decibels lower for frequencies in the lower range (e.g., 200 Hertz) and progressively increase the initial lower decibel level as the frequency increases until 10,000 Hertz when users typically do not experience hyperacusis. Similarly, phonophobia is a fear or emotional reaction to certain sounds. If a user identifies that they have phonophobia, the frequency module 306 may instruct the auditory device to skip sounds that the user identifies as problematic.

In some embodiments, the user interface module 302 generates graphical data for displaying a user interface to select from two or more levels of granularity for numbers of listening bands for the threshold-level testing and/or the frequency gain balance testing. In some embodiments, the user selects a level of granularity that applies to both the threshold-level testing and the frequency gain balance testing. In some embodiments, the user interface may include radio buttons for selecting a particular number of listening bands or a field where the user may enter a number of listening bands or whether one or fewer octaves are represented by a band.

FIG. 4B is an example user interface 425 for selecting a level of granularity of the hearing test. In this example, the user interface 425 includes three levels: rough, which may include a band for each octave; middle, which may include a band for each ⅓ octave; and fine, which may include a band for each ⅙ octave. The user may select one of the three buttons 430, 435, 440 to request the corresponding level of granularity.

In some embodiments, the user interface module 302 includes an option for selecting a type of background noise for the threshold-level testing and/or the frequency gain balance testing. The background noise may include white noise, voices, music, and various combinations of environmental background noise to the different hearing tests. In some embodiments, only one type of background noise is used. In some embodiments, all types of background noises are used. In some embodiments, the user interface module 302 includes an option for increasing a decibel level of background noise.

During the threshold-level testing, in some embodiments the user interface module 302 generates graphical data for displaying a user interface with a way for the user to identify when the user hears a sound. For example, the user interface may include a button that the user can select to confirm that the user hears a sound. In another example, the user interface may include a slider for increasing the volume of a sound until the user can hear the sound.

During the frequency gain balance testing, in some embodiments the user interface module 302 generates graphical data for displaying a user interface for the user to identify when a first test sound and a second test sound are perceived as being played at the same volume.

FIG. 4C is an example user interface 450 for frequency gain balance testing. The frequency module 306 instructs the audio device to generate test sound A and test sound B for the listening bands where test sound A is the reference test sound. The user interface 450 includes a slider 455 for changing the decibel level of test sound B. Once test sound A and test sound B sound the same to the user, the user may select the done button 460. The user may press the test sound A button 453 to hear test sound A again to compare it to test sound B. Once the user is finished with test sound A and test sound B, the frequency module 306 may advance to the next band in the set of listening bands being tested.

During the speech-clarity testing, in some embodiments the user interface module 302 generates graphical data for displaying a user interface for the user to identify which factors make speech sound the clearest. For example, the user interface may include radio buttons or sliders for changing different variables, such as a volume of background noise, a volume of the people speaking, and a volume of different factors including consonant grouping. This helps identify words or sound combinations that the user may have difficulty hearing.

Turning to FIG. 4D, an example user interface 475 is illustrated for speech-clarity testing. In this example, the auditory device plays a speaking test of a male voice. If the user is not satisfied with how the speaking test sounds, the user may select different sliders for adjusting the frequencies. In this example, the user adjusted a first slider 476 to have a frequency of 5 kHz, a second slider to have a frequency of 3 kHz, a third slider to have a frequency of 1 kHz, and a fourth slider to have a frequency of 500 Hz. The user may be better able to understand sounds when the frequencies are adjusted. A different number of sliders may be used. For example, the user interface 475 may include a minimum of two sliders for adjusting the high frequencies and the middle frequencies.

In some embodiments, the speaking test may repeat until the user is satisfied with the speaking test and selects the next button 488. The speaking test may also include background noise where the speaking test loops for each background noise setting. Each setting may have the same type of background noise or each setting may be different.

In some embodiments, the user starts the speaking test with a female voice by selecting the female button 486. The user may return to the speaking test with the male voice by selecting the male button 484. In some embodiments, the user does not have the option of switching between voices until all the background noises have been played.

In some embodiments, the user interface module 302 may generate graphical data for displaying a user interface that allows a user to repeat the hearing tests. For example, the user may feel that the results are inaccurate and may want to test their hearing to see if there has been an instance of hearing loss that was not identified during testing. In another example, a user may experience a change to their hearing conditions that warrant a new test, such as a recent infection that may have caused additional hearing loss.

In some embodiments, the user interface module 302 generates graphical data for displaying a user interface for determining user preferences for generating one or more presets, the specifics of which will be described in greater detail below with reference to the preset module 312. In some embodiments, the user preferences are determined after the hearing tests are completed. For example, after the speech-clarity testing is completed, the user interface module 302 may generate a user interface with questions about whether the user prefers the use of a noise cancellation preset or an ambient noise preset in situations where people are speaking, such as during telephone calls.

In yet another example, after the speech test is completed, the user interface module 302 may generate a user interface with questions about speech preferences, such as whether the user prefers a voice in a crowded room preset or a type of speech. For example, the auditory device may play different settings that are possible for hearing voices in a crowded room. A first preset may reduce background noise and amplify voices and a second preset may reduce background noises and voices except for a voice closest to the user, etc. The user interface may include a volume slider to adjust the volume of the sound and a sound slider to allow the user to hear different presets. The user can select the button when the user is satisfied with the preset. In another example, the user interface could include two sound sliders, such as a first sound slider for modifying the background noise and a second sound slider for modifying the voices.

Other user interfaces may be used to determine the one or more presets. For example, instead of using a slider to change the types of background noises, the user interface module 302 may generate a user interface that cycles through different situations and the user interface includes a slider for changing the decibel level or there may be no slider and instead the user preferences are determined with radio buttons, confirmation buttons, icons, vocal responses from the user, etc.

In some embodiments, the user interface module 302 generates graphical data for a user interface that includes icons for different presets that allows the user to modify the one or more presets. For example, the user interface may include an icon and associated text for a noise cancellation preset, an ambient noise preset, a speech and music preset, a type of noise preset, and a type of auditory condition. The type of noise preset may include individual icons for presets corresponding to each type of noise, such as one for construction noise and another for noises at a particular frequency. The type of auditory condition preset may include individual icons for presets corresponding to each type of auditory condition, such as an icon for tinnitus and an icon for phonophobia.

In some embodiments, the user interface module 302 generates graphical data for displaying a user interface that includes an option to override the one or more presets. For example, continuing with the example above, the user interface may include icons for different presets and selecting a particular preset causes the user interface to display information about the particular preset. For example, selecting the ambient noise preset may cause the user interface to show that the ambient noise preset is automatically on. The user may provide feedback, such as turning off the ambient noise preset so that it is automatically off. The preset module 312 may update the one or more presets based on the feedback from the user.

The threshold module 304 implements a threshold-level testing. In some embodiments, the threshold module 304 includes a set of instructions executable by the processor 335 to implement the threshold-level testing. In some embodiments, the threshold module 304 is stored in the memory 337 of the computing device 300 and can be accessible and executable by the processor 335.

In some embodiments, the threshold-level testing includes testing pink-band levels. Pink noise is a category of sounds that contains all the frequencies that a human ear can hear. Specifically, pink noise contains the frequencies from 20 Hertz to 20,000 Hertz. Although humans may be able to discern that range of frequencies, humans hear the higher frequencies less intensely. By testing the complete range of frequencies, pink-band level testing advantageously detects the full range of human hearing. Conversely, some traditional hearing tests stop testing after some frequencies in response to a user experiencing hearing loss at a particular frequency. Traditional hearing tests may miss the fact that certain hearing conditions only affect certain frequencies. For example, tinnitus may affect hearing sensitivity in frequencies between 250-16,000 Hertz but does not necessarily affect all those frequencies. As a result, if a user experiences hearing loss at 4,000 Hertz due to tinnitus, the user may not have any hearing loss at 8,000-16,000 Hertz, which would be missed by a traditional hearing test.

Other types of noise may be used for threshold-level testing. For example, instead of pink-noise levels, the threshold module 304 may use white-noise levels or brown-noise levels.

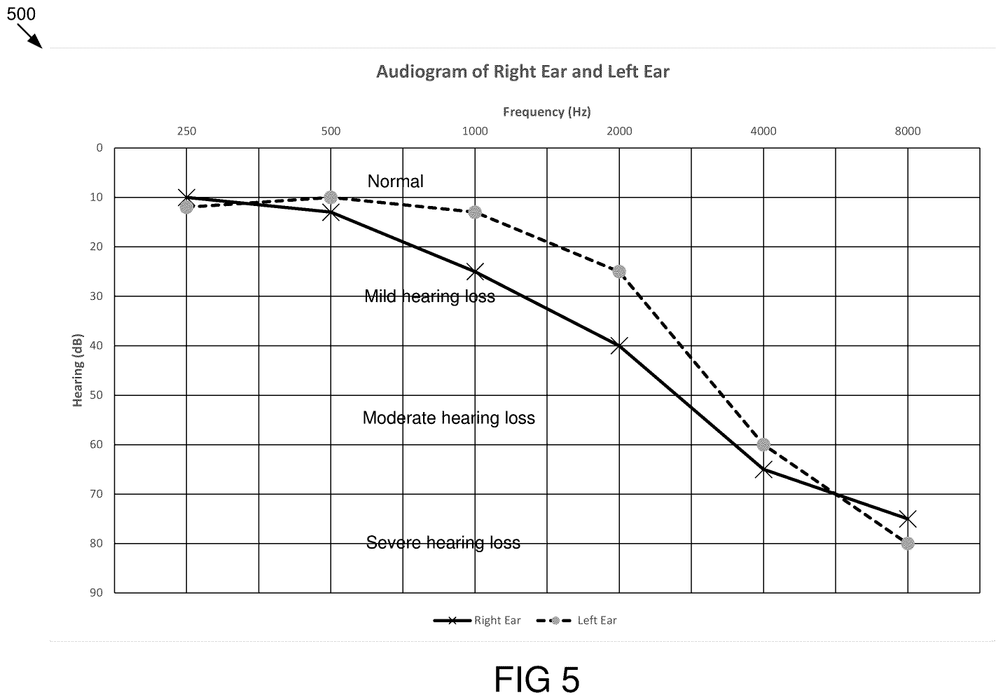

Hearing may be tested in bands that span across different frequencies. Bands are like stereo equalizers. They control volume for different frequencies because a user may need higher volume for one band but not another. For example, FIG. 5 is an illustration of an example audiogram 500 of a right ear and a left ear. In this example, the hearing is tested using six frequency bands: 250 Hertz, 500 Hertz, 1000 Hertz, 2000 Hertz, 4000 Hertz, and 8000 Hertz. People may experience different levels of hearing loss depending on the frequencies. In this example, the left and right ears experience normal hearing until 1000 Hertz when the right ear experiences mild hearing loss where a hearing aid would need to add 20 decibels of gain to reach normal hearing. At 2000 Hertz the left ear experiences mild hearing loss and the right ear experiences between mild and moderate hearing loss. At 4000 Hertz both ears experience moderate hearing loss and the hearing aid would need to add 45 decibels of gain to reach normal hearing. At 8000 Hertz when both ears experience severe hearing loss.

In some embodiments, the threshold module 304 tests users at different levels of granularity in the frequency range between bands based on a user selection. For example, the user may be provided with the option of a rough test, a middle test, and a fine test. The rough test may use bands for every octave. This may prevent a user from getting annoyed with excessive testing.

In some embodiments, the threshold module 304 may employ rough testing until the user identifies frequencies where the user's hearing is diminished and, at that stage, the threshold module 304 implements more narrow band testing. For example, the threshold module 304 may test every octave band until the user indicates that they cannot hear a sound in a particular band or the sound is played at a higher decibel level to be audible to the user for the particular band. At that point, the threshold module 304 may implement band testing below and above the particular band at intervals of one twelfth octave bands to further refine the extent of the user's hearing loss. In some embodiments, if the user experiences hearing loss in the lower frequencies, such as below 1000 Hertz, the threshold module 304 may test in smaller bandwidths than for the higher frequencies.

In some embodiments, the threshold module 304 implements pink noise band testing by playing a test sound at a listening band, where the intervals for the listening bands may be based on the different factors discussed above. The threshold module 304 determines whether a confirmation was received that the user heard the test sound. If the threshold module 304 did not receive the confirmation that the user heard the test sound, the threshold module 304 may instruct the auditory device to increase the decibel level of the test sound until the confirmation is received or the test sound is played at a decibel level that meets a decibel threshold. For example, the decibel level may start at 0 decibels and the decibel threshold may be 85 decibels. Responsive to receiving the confirmation that the user heard the test sound or the test sound was played at the decibel threshold, the threshold module 304 may advance the listening band so that N=N+1 until the listening band N meets a total listening band. For example, the threshold module 304 may continue until N is greater than 20,000 Hertz. During each step or at the conclusion of the threshold-level testing, the threshold module 304 updates the hearing profile with the test results.

In some embodiments, the threshold module 304 instructs the auditory device to play a background noise with the test sound. The background noise may be white noise, voices, music, or any combination of white noise, voices, and music. In some embodiments, the user may select a decibel level at which the background noise is played.

In some embodiments, the threshold module 304 implements testing on a first ear and then on a second ear (e.g., first the left ear and then the right ear or first the right ear and then the left ear) and generates different hearing profiles for each ear.

The frequency module 306 implements frequency gain balance testing. In some embodiments, the frequency module 306 includes a set of instructions executable by the processor 335 to implement the frequency gain balance testing. In some embodiments, the frequency module 306 is stored in the memory 337 of the computing device 300 and can be accessible and executable by the processor 335.

In some embodiments, the frequency module 306 tests users at different levels of granularity in the frequency range between bands based on a user selection. For example, the user may be provided with the option of a rough test, a middle test, and a fine test. The intervals for the listening bands are based on the levels of granularity.

In some embodiments, the frequency module 306 implements the frequency gain balance testing by determining a type of equal-loudness contour. The frequency module 306 instructs the auditory device to play a first test sound at listening band N and a second test sound at listening band N+1. The listening bands may include pink noise band testing, as described in greater detail above, or other types of noise (e.g., white noise or brown noise). For example, the first test sound may be at a frequency corresponding to a first octave and the second test sound may be a frequency corresponding to a second octave. The first test sound and the second test sound may be played at different decibel levels because hearing loss or the difference in how frequencies are perceived may cause the user to perceive the test sounds differently. In some embodiments, the frequency module 306 may not test sounds at particular frequencies if the threshold module 304 determined that the user cannot hear sounds at those particular frequencies.

The first test sound functions is played at a decibel level that is slightly higher than the threshold decibel level established by the threshold module 304 for the particular frequency. For example, if the user experiences no hearing loss, the reference test sound may be played at 65 decibels sound pressure level (SPL) because 65 SPL is about the loudness at which people speak. SPL is a decibel scale that is defined relative to a reference that is approximately the intensity of a 1000 Hertz sinusoid that is just barely audible to the user.

The frequency module 306 determines whether a confirmation was received that the first test sound and the second test sound were perceived to be a same volume. In some embodiments, the user interface module 302 generates a user interface that asks the user if the first test sound and the second test sound were perceived to be played at the same volume. The user may not respond until the test sounds are perceived to be at the same volume or the user may explicitly state that the test sounds are perceived to be at different volumes. If the frequency module 306 does not receive the confirmation, the frequency module 306 raises a decibel level of the second test sound until the first test sound and the second test sound are perceived to be the same volume.

If the frequency module 306 determines that the user confirmed that the first test sound and the second test sound are perceived to be the same volume, the frequency module 306 advances the listening bands so that N=N+1. The frequency module 306 determines if the listening band N meets a total listening band. For example, the threshold module 304 may continue until N is greater than 20,000 Hertz.

If the listening band N does not meet the total listening band, the frequency module 306 repeats the previous steps and plays the first test sound at listening band N and the second test sound at listening band N until the listening band N meets the total listening band. The first test sound functions as a reference test sound such that the second test sound is modified to match the perceived volume of the first test sound. When the frequency module 306 advances the listening bands, the second test sound becomes the reference test sound to a third test sound at listening band N+1.

If the listening band N meets a total listening band, the frequency module 306 updates the hearing profile. The frequency module 306 may also update the hearing profile periodically, after each step, etc.

The following is an example scenario for illustration. The frequency module 306 instructs the auditory device to play a first test sound at listening band 500 Hz that is played at 65 decibels SPL and a second test sound that is an octave higher at listening band 1000 Hz that is played at 85 decibels SPL because threshold-level hearing test indicated that the user cannot hear sounds at 1000 Hz that are played lower than 20 decibels SPL so 85 decibels SPL is approximately the level needed for the user to have conversations at 1000 Hz. The user perceives the test sounds as being different until the second test sound is increased to 87 decibels SPL.

Next, the frequency module 306 advances the listening bands so that the second test sound is at 1000 Hz and the third test sound is at 2000 Hz. The frequency module 306 instructs the auditory device to play the second test sound at 87 decibels SPL because the second test sound is now the reference test sound that the third test sound is compared against. The frequency module 306 instructs the auditory device to play the third test sound at 75 decibels SPL because the threshold-level hearing test indicated that the user cannot hear sounds at 2000 Hz that are played lower than 10 decibels SPL. The frequency module 306 may continue this process until the listening band N is at 20,000 Hz and 20,000 Hz meets the total listening band.

In some embodiments, the frequency module 306 instructs the auditory device to play a background noise with the test sound. The background noise may be white noise, voices, music, or any combination of white noise, voices, and music. In some embodiments, the user may select a decibel level at which the background noise is played.

In some embodiments, the frequency module 306 implements testing on a first ear and then on a second ear (e.g., first the left ear and then the right ear or first the right ear and then the left ear) and generates different profiles for each ear.

The speech module 308 implements a speech test. In some embodiments, the speech module 308 includes a set of instructions executable by the processor 335 to implement the speech test. In some embodiments, the speech module 308 is stored in the memory 337 of the computing device 300 and can be accessible and executable by the processor 335.

In some embodiments, the speech module 308 implements the speech test by instructing the auditory device to play different combinations of male speech and female speech. For example, the speech module 308 may instruct the auditory device to play a speaking test with a voice of a first gender (e.g., male speech), complete the speech test, and then instruct the auditory device to play the speaking test with a voice of a different gender (e.g., female speech).

In some embodiments, the speech module 308 implements speech testing by instructing the auditory device to play a speaking test. The speech module 308 may also instruct the auditory device to play the speaking test with a background noise. In some embodiments, the speech module 308 instructs the auditory device to play the test sound at a predetermined SPL, such as 65 decibels SPL. In some embodiments, the speech module 308 instructs the auditory device to play the test sound a predetermined level (e.g., 40 decibels) above the softest level at which the user begins to recognize speech or the tones from the pink band testing.

The speech module 308 determines whether confirmation was received that the user is satisfied with the speaking test. For example, the user interface module 302 may generate a user interface with an option to move to a subsequent test when the user is satisfied and if not, to use two or more sliders to modify how the speaking test sounds. For example, a first slider may be used to adjust the higher frequencies (i.e., 3,000-5,000 Hz) to better understand certain consonants like K, F, S, ST, TH, etc. A second slider may be used to adjust the middle frequencies (i.e., 500-2,000 Hz) to better understand vowel-type sounds like B, P, A, H, SH, CH, etc.

In some embodiments, the speech module 308 instructs the auditory device to play a background noise with the test sound. The background noise may be white noise, voices, music, or any combination of white noise, voices, and music. In some embodiments, the user may select a decibel level at which the background noise is played. Once all the background noises have been played and the user is satisfied with the speaking test, the user may have the option to play the speaking test with a different gender. In some embodiments, once both genders have been played, or the user only wants to take the test with a speaking test played with one type of voice, the hearing profile is updated. In some embodiments, the speech module 308 updates the hearing profile after each step.

In some embodiments, the speech module 308 implements testing on a first ear and then on a second ear (e.g., first the left ear and then the right ear or first the right ear and then the left ear) and generates different profiles for each ear.

The profile module 310 generates and updates a hearing profile associated with a user. In some embodiments, the profile module 310 includes a set of instructions executable by the processor 335 to generate the hearing profile. In some embodiments, the profile module 310 is stored in the memory 337 of the computing device 300 and can be accessible and executable by the processor 335.

The profile module 310 generates a hearing profile based on the threshold-level testing, the frequency gain balance testing, and the speech-clarity testing. In some embodiments, the profile module 310 updates the hearing profile periodically (e.g., every minute, every five minutes), every time a sound is confirmed, or every time a test is completed. In some embodiments, the profile module 301 maintains separate profiles for each type of auditory device. For example, the profile module 301 generates a first hearing profile for headphones and a second hearing profile for speakers.

In some embodiments, the profile module 310 receives an audiometric profile from the server and compares the hearing profile to the audiometric profile in order to make recommendations for the user. In some embodiments, the profile module 310 modifies the hearing profile to include instructions for producing sounds based on a comparison of the hearing profile to the audiometric profile. For example, the profile module 310 may identify that there is a 10-decibel hearing loss at 400 Hertz based on comparing the hearing profile to the audiometric profile and the hearing profile is updated with instructions to produce sounds by increasing the auditory device by 10 decibels for any noises that occur at 400 Hertz.

The preset module 312 generates one or more presets that correspond to a user preference. In some embodiments, the preset module 312 includes a set of instructions executable by the processor 335 to generate the one or more presets. In some embodiments, the preset module 312 is stored in the memory 337 of the computing device 300 and can be accessible and executable by the processor 335.

In some embodiments, the preset module 312 assigns one or more default presets. The one or more default presets may be based on the most common presets used by users. In some embodiments, the one or more default presets may be based on the most common presets used by users of a particular demographic (e.g., based on sex, age, similarity of user profiles, etc.). The preset module 312 may implement testing to determine user preferences that correspond to the one or more presets or the preset module 312 may update the one or more default presets in response to receiving feedback from the user.

The preset module 312 generates one or more presets that modify settings established in the hearing profile. In some embodiments, the profile module 310 generates a hearing profile for a first type of auditory device and the preset module 312 generates a preset for a second type of auditory device. For example, the hearing profile may be generated based on tests for a laptop speaker. The preset module 312 may determine a preset for earbuds that modifies the settings established by the hearing profile. For example, the decibel level is decreased for the earbuds since they are closer to the ear than a laptop speaker.

The preset module 312 determines one or more presets that correspond to a user preference. For example, the presets include a noise cancellation preset, an ambient noise preset, a speech and music preset, a music in a room preset, a voice in a crowded room preset, a type of enclosure, a type of speech, a type of music, a type of noise, a type and model of auditory device, and/or a type of auditory condition.

The noise cancellation preset removes external noise from the auditory device. For example, the auditory device may include microphones that detect sounds and speakers that emit signals that cancel out the noise frequencies to cancel out both sets of sounds when the soundwaves from the noise and the signals collide. In some embodiments, the preset module 312 determines that the user prefers the noise cancellation preset and, as a result, the noise cancellation preset is automatically used. In some embodiments, the noise cancellation preset is applied to particular situations. For example, the preset module 312 may determine that the user wants the noise cancellation preset to be activated when the user enters a crowded room, but not when the user is in a quiet room or in a vehicle.

The ambient noise preset causes the auditory device to provide a user with surrounding outside noises while also playing other sounds, such as music, a movie, etc. The auditory device may include microphones that detect the outside noises and provide the outside noises to the user with speakers. In some embodiments, the preset module 312 determines that the user prefers the ambient noise preset and, as a result, the ambient noise preset is automatically used. In some embodiments, the ambient noise preset is applied to particular situations. For example, the preset module 312 may determine that the user wants the ambient noise preset to be activated when the user is outside (such as if the user is running), but not when the user is inside an enclosure (such as a room or a vehicle).

In some embodiments, the preset module 312 generates a noise cancellation and ambient noise preset that may cause the auditory device to provide a user with noise cancellation of noises that are not directly surrounding the user while allowing in sounds that directly surround the user through the ambient noise aspect of the preset. In some examples, the noise cancellation and ambient noise preset includes three options: a first setting activates the ambient noise function and the noise cancellation function, a second setting turns off the noise-cancellation function so only the ambient noise function is active, and a third setting turns off the ambient noise function so only the noise cancellation function is activated.

In some embodiments, the preset module 312 identifies a speech and music preset that combines user preferences for speech and music or separately identifies a speech preset and a music preset. The speech preset may include a variety of different user preferences relating to speech. For example, during speech band testing, the preset module 312 may identify that the user has difficulty hearing certain sounds in speech, such as words that begin with “th” or “sh.” As a result, the speech preset may include amplification of words that use those particular sounds.

The music preset may include a variety of different user preferences relating to music. For example, the user may identify that there are certain frequencies or situations during which the user experiences hypersensitivity. For example, the user may identify a particular frequency that causes distress; or a particular action that bothers a user (such as construction noises) or based on a particular condition like misophonia (such as chewing or sniffing noises).

In yet another example, the preset module 312 may determine that a user prefers equalizer settings to be activated. Equalizers are software or hardware filters that adjust the loudness of specific frequencies. Equalizers work in bands, such as treble bands and bass bands, which can be increased or decreased. As a result of applying equalizer settings, the user may hear all frequencies with the same perceived loudness based on adjusting the decibel levels based on the music testing.

In some embodiments, the presets may include more specific situations, such as a music in a room preset that causes the auditory device to apply different music settings in a room based on user preferences. The advantage to having this more specific presets is that it may be easier for a user to modify the specific preset for music in a room than having to repeat the entire process of identifying user preferences in order to modify this one particular preference. Similarly the presets may include a voice in a crowded room preset because a user may have particular difficulty with hearing voices in a crowded room, but may not struggle with other types of background noise. As a result, the user may want the voice in a crowded room preset to be active, but not want the noise cancellation preset to be automatically activated.

In some embodiments, the presets may be even more specific and include a preset for a type of enclosure, a type of speech, a type of music, a type of noise, a type and model of auditory device, and/or a type of auditory condition. The type of enclosure may include a small room (e.g., an office), a medium room (e.g., a restaurant), a large room (e.g., a conference hall), a car, etc. The type of speech may include particular words or sounds that the user has difficulty hearing and, as a result, are amplified. The type of music may include particular instruments (e.g., a preference to avoid shrill sounds, such as a violin) or music genres (e.g., a preference to avoid playing music with deep base unless the decibel level for the base is reduced).

In some embodiments, the preset module 312 receives feedback from a user. The user may provide user input to a user interface that changes one or more presets. For example, the user may change a preset for a type of enclosure for a vehicle to automatically apply noise cancellation to the road noise and amplify voices inside the vehicle. The preset module 312 updates the one or more presets based on the feedback. For example, the preset module 312 may change the preset for the type of enclosure from off to on. In some embodiments, the preset module 312 does not change the one or more presets until a threshold amount of feedback has been received. For example, the preset module 312 may not change a preset until the user has changed the preset a threshold of four times (or three, five, etc.).

The profile module 310 transmits the hearing profile and/or the preset module 312 transmits the one or more presets to the auditory device and/or a server for storage via the I/O interface 339.

### Example Methods

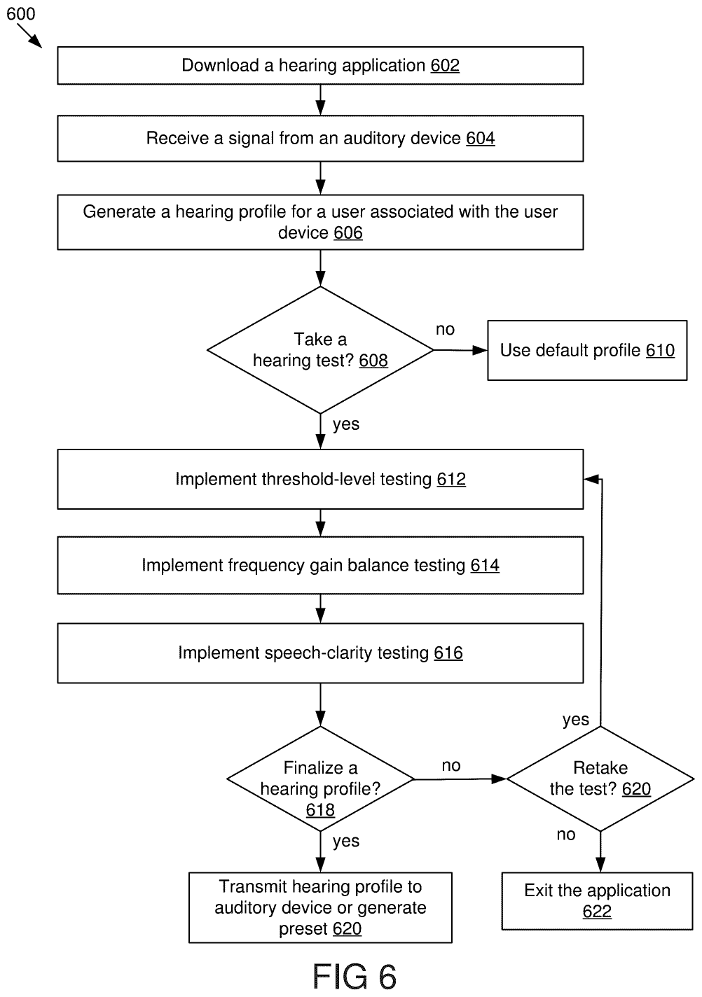

FIG. 6 illustrates a flowchart of a method 600 to implement a hearing test according to some embodiments described herein. The method 600 may be performed by the computing device 300 in FIG. 3. For example, the computing device 300 may be the user device 115 or the auditory device 120 illustrated in FIG. 1. The computing device 300 includes a hearing application 103 that implements the steps described below.

In embodiments where the method 600 is performed by the user device 115 in FIG. 1, the method 600 may start with block 602. At block 602, a hearing application is downloaded. In embodiments where the method 600 is performed by the auditory device 120, the method may start with block 606. Block 602 may be followed by block 604.

At block 604, a signal is received from an auditory device. For example, the signal may be for establishing a Bluetooth connection with a user device. Block 604 may be followed by block 606.

At block 606, a hearing profile is generated for a user associated with the user device. For example, the user profile includes the user's name, demographic information, etc. Block 606 may be followed by block 608.

At block 608, it is determined whether the user wants to take a hearing test. If the user does not want to take a hearing test, block 608 is followed by block 610. At block 610, a default profile is used. If the user does want to take a hearing test, block 608 is followed by block 612.

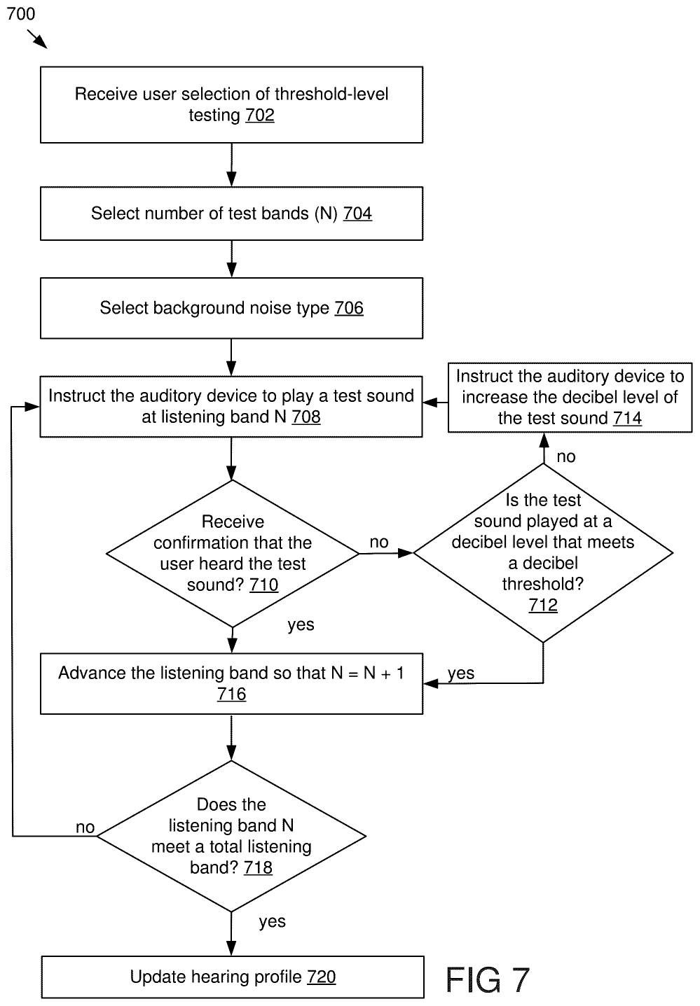

At block 612, threshold-level testing is implemented. For example, the threshold-level testing may include the method 700 described in FIG. 7. Block 612 may be followed by block 614.

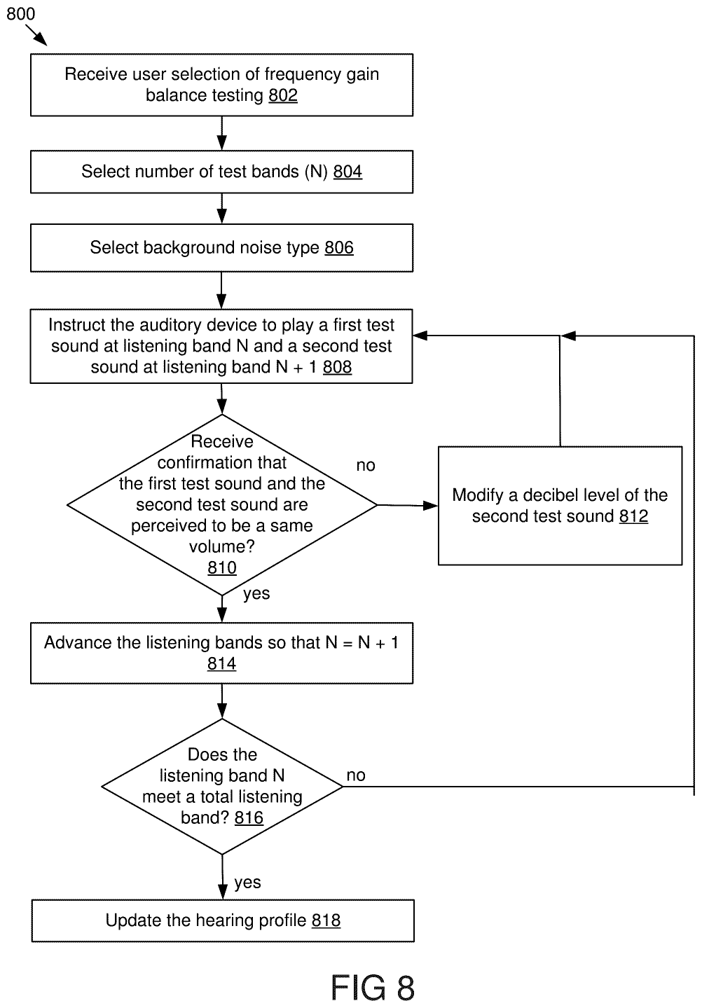

At block 614, frequency gain balance testing is implemented. For example, the frequency gain balance testing may include the method 800 described in FIG. 8. Block 614 may be followed by block 616.

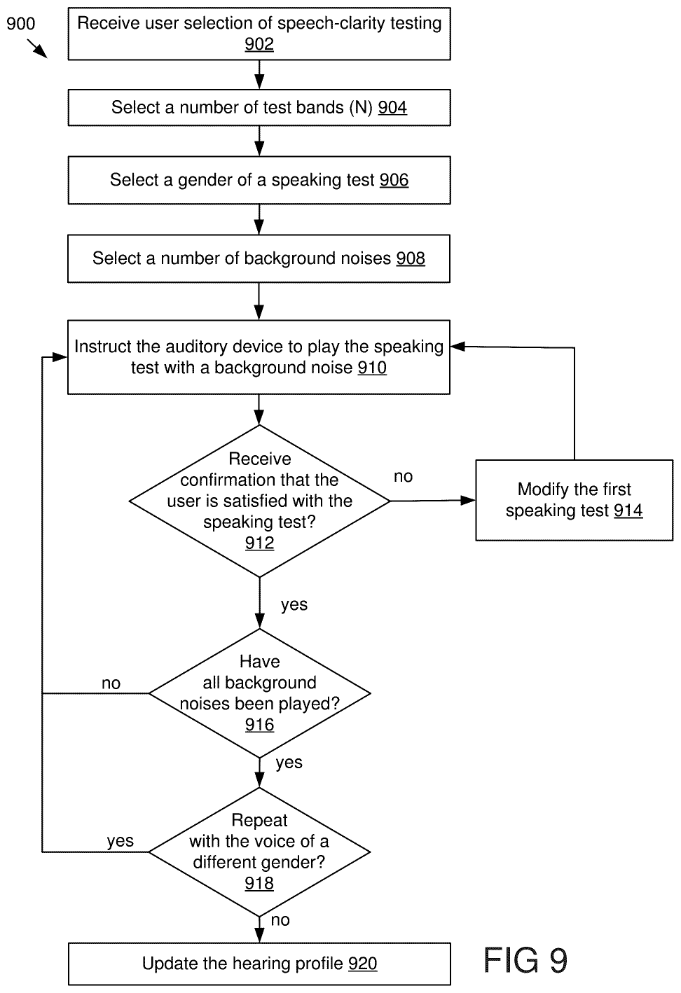

At block 616, speech-clarity testing is implemented. For example, the speech-clarity testing may include the method 900 described in FIG. 9. Block 616 may be followed by block 618.

At block 618, it is determined whether a hearing profile is to be finalized. For example, the hearing application 103 may instruct the auditory device to play music, stream a television show, etc. to help the user determine if they are satisfied with the hearing profile. If the user wants the hearing profile to be generated, block 618 may be followed by block 620. At block 620, the hearing profile is transmitted to the auditory device or a preset is generated. If the user does not want the hearing profile to be generated, block 618 may be followed by block 620. At block 620, it is determined whether to retake the test. If the user wants to retake the test, block 620 may be followed by block 612 where the tests begin again. If the user does not want to retake the test, block 620 may be followed by block 622. At block 622, the application is exited.

FIG. 7 illustrates a flowchart of a method 700 to implement threshold-level testing according to some embodiments described herein. The method 700 may be performed by the computing device 300 in FIG. 3. For example, the computing device 300 may be the user device 115 or the auditory device 120 illustrated in FIG. 1. The computing device 300 includes a hearing application 103 that implements the steps described below.

The method 700 may start with block 702. At block 702, user selection of threshold-level testing is received. Block 702 may be followed by block 704.

At block 704, a number of test bands (N) are selected. Block 704 may be followed by block 706.

At block 706, a background noise type may be selected. For example, the background noise may include white noise, voices, music, or a combination of the types of background noise. Block 706 may be followed by block 708.

At block 708, the auditory device is instructed to play a test sound at listening band N. Block 708 may be followed by block 710.

At block 710, it is determined whether confirmation is received that the user heard the test sound. For example, the user may select an icon on a user interface when the user hears a test sound. If the confirmation is not received, block 710 may be followed by block 712. At block 712, it is determined whether the sound played at a decibel level meets a decibel threshold. For example, the decibel threshold may be 110 decibels because after 110 decibels the sound may cause hearing damage to the user. If the sound does not meet the decibel threshold, block 712 may be followed by block 714. At block 714, the auditory device is instructed to increase the decibel level of the test sound. Block 714 may be followed by block 708. If the test sound is played at a decibel level that meets a decibel threshold, block 712 may be followed by block 716.

If confirmation is received that the user heard the test sound, block 710 may be followed by block 716. At block 716, the listening band is advanced so that N=N+1. Block 716 may be followed by block 718.

At block 718, it is determined whether the listening band N meets a total listening band. If the listening band N does not meet the total listening band, block 718 may be followed by block 708. If the listening band N does meet the total listening band, block 718 may be followed by block 720.

At block 720, the hearing profile is updated. The hearing profile may be stored locally on the user device 115 or the auditory device 120 in FIG. 1 and/or on the server 101 in FIG. 1.

FIG. 8 illustrates a flowchart of a method 800 to implement frequency gain balance for music according to some embodiments described herein.

At block 802, user selection of frequency gain balance testing is received. This step may be an optional step and instead the end of threshold-level testing may automatically lead to the frequency gain balance testing. Block 802 may be followed by block 804.

At block 804, a number of test bands (N) are selected. Block 804 may be followed by block 806.

At block 806, a background noise type may be selected. For example, the background noise may include white noise, voices, music, or a combination of the types of background noise. Block 806 may be followed by block 808.

At block 808, the auditory device is instructed to play a first test sound at listening band N and a second test sound at listening band N+1. Block 808 may be followed by block 810.

At block 810, it is determined whether confirmation is received that the first test sound and the second test sound are perceived to be a same volume. If the first test sound and the second test sound are not confirmed to be a same volume, block 810 may be followed by block 812. At block 812, a decibel level of the second test sound is modified. Block 812 may be followed by block 808.

If the first test sound and the second test sound are confirmed to be a same volume, block 810 may be followed by block 814. At block 814, the listening bands are advanced so that N=N+1. Block 814 may be followed by block 816.

At block 816, it is determined whether the listening band N meets a total listening band. If the listening band N does not meet a total listening band, block 816 may be followed by block 808.

If the listening band N meets a total listening band, block 816 may be followed by block 818. At block 818, the hearing profile is updated. The hearing profile may be stored locally on the user device 115 or the auditory device 120 in FIG. 1 and/or on the server 101 in FIG. 1.

FIG. 9 illustrates a flowchart of a method 900 to implement speech clarity according to some embodiments described herein. The method 900 may be performed by the computing device 300 in FIG. 3. For example, the computing device 300 may be the user device 115 or the auditory device 120 illustrated in FIG. 1. The computing device 300 includes a hearing application 103 that implements the steps described below.

At block 902, user selection of speech-clarity testing is received. This step may be an optional step and instead the end of threshold-level testing or the end of frequency gain balance testing may automatically lead to the speech clarity testing. Block 902 may be followed by block 904.

At block 904, a number of test bands (N) are selected. For example, the hearing application 103 may receive a selection of a number of test bands from the user via a user interface. Block 904 may be followed by block 906.

At block 906, a gender of the speaking test is selected. For example, the hearing application 103 may receive a selection of a gender of a speaking test, such as female or male. Block 906 may be followed by block 908.

At block 908, a number of background noises are selected. The hearing application 103 may receive a selection of the number of background noises via a user interface where the number is 0, 1, 2, 3, etc. For example, the background noise may include white noise, voices, music, or a combination of the types of background noise. Block 908 may be followed by block 910.

At block 910, the auditory device is instructed to play the speaking test with a background noise. For example, the background noise may be nothing because the user selected to not include a background noise. In another example, the background noise may be part of a set of background noises and the background noise may change each time it is played with the speaking test. Block 910 may be followed by block 912.

At block 912, it is determined whether confirmation is received that the user is satisfied with the speaking test. For example, if the user is satisfied with the speaking test, the user may select a done button on a user interface.

If confirmation is not received that the user is satisfied with the speaking test, block 912 may be followed by block 914. At block 914, the first speaking test is modified. For example, responsive to the user moving one or more sliders on a user interface, the hearing application 103 may change how consonant groupings sound for different frequencies. Block 914 may be followed by block 910.

If confirmation is received that the user is satisfied with the speaking test, block 912 may be followed by block 916.

At block 916, it is determined whether all background noises have been played. If all background noises have not been played, block 916 may be followed by block 910. For example, if the user selects two background noises and the speaking test was played with the first background noise, the speaking test may be played again with the second background noise. If all background noises have been played, block 916 may be followed by block 918.

At block 918, it is determined whether to repeat the speech-clarity testing with the voice of a different gender. For example, the user may select a male button on the user interface once the user is satisfied with the speaking test spoken with a female voice. If the speech-clarity testing is repeated with the different gender, block 918 may be followed by block 910.

If the speech-clarity testing is not repeated with the voice of a different gender, block 918 may be followed by block 920. At block 920, the hearing profile is updated. The hearing profile may be stored locally on the user device 115 or the auditory device 120 in FIG. 1 and/or on the server 101 in FIG. 1.

Although the description has been described with respect to particular embodiments thereof, these particular embodiments are merely illustrative, and not restrictive.

Any suitable programming language can be used to implement the routines of particular embodiments including C, C++, Java, assembly language, etc. Different programming techniques can be employed such as procedural or object oriented. The routines can execute on a single processing device or multiple processors. Although the steps, operations, or computations may be presented in a specific order, this order may be changed in different particular embodiments. In some particular embodiments, multiple steps shown as sequential in this specification can be performed at the same time.

Particular embodiments may be implemented in a computer-readable storage medium for use by or in connection with the instruction execution system, apparatus, system, or device. Particular embodiments can be implemented in the form of control logic in software or hardware or a combination of both. The control logic, when executed by one or more processors, may be operable to perform that which is described in particular embodiments.

Particular embodiments may be implemented by using a programmed general purpose digital computer, by using application specific integrated circuits, programmable logic devices, field programmable gate arrays, optical, chemical, biological, quantum or nanoengineered systems, components and mechanisms may be used. In general, the functions of particular embodiments can be achieved by any means as is known in the art. Distributed, networked systems, components, and/or circuits can be used. Communication, or transfer, of data may be wired, wireless, or by any other means.

It will also be appreciated that one or more of the elements depicted in the drawings/figures can also be implemented in a more separated or integrated manner, or even removed or rendered as inoperable in certain cases, as is useful in accordance with a particular application. It is also within the spirit and scope to implement a program or code that can be stored in a machine-readable medium to permit a computer to perform any of the methods described above.

A “processor” includes any suitable hardware and/or software system, mechanism or component that processes data, signals or other information. A processor can include a system with a general-purpose central processing unit, multiple processing units, dedicated circuitry for achieving functionality, or other systems. Processing need not be limited to a geographic location, or have temporal limitations. For example, a processor can perform its functions in “real time,” “offline,” in a “batch mode,” etc. Portions of processing can be performed at different times and at different locations, by different (or the same) processing systems. Examples of processing systems can include servers, clients, end user devices, routers, switches, networked storage, etc. A computer may be any processor in communication with a memory. The memory may be any suitable processor-readable storage medium, such as random-access memory (RAM), read-only memory (ROM), magnetic or optical disk, or other non-transitory media suitable for storing instructions for execution by the processor.

As used in the description herein and throughout the claims that follow, “a”, “an”, and “the” includes plural references unless the context clearly dictates otherwise. Also, as used in the description herein and throughout the claims that follow, the meaning of “in” includes “in” and “on” unless the context clearly dictates otherwise.

Thus, while particular embodiments have been described herein, latitudes of modification, various changes, and substitutions are intended in the foregoing disclosures, and it will be appreciated that in some instances some features of particular embodiments will be employed without a corresponding use of other features without departing from the scope and spirit as set forth. Therefore, many modifications may be made to adapt a particular situation or material to the essential scope and spirit.

We claim:

1. A computer-implemented method performed on a user device, the method comprising:
receiving a signal from an auditory device;
determining whether a user selected to take a hearing test;
implementing threshold-level testing;
implementing frequency gain balance testing;
implementing speech-clarity testing; and
generating a hearing profile based on at least one selected from the group of the threshold-level testing, the frequency gain balance testing, the speech-clarity testing, and combinations thereof.

2. The computer-implemented method of claim 1, further comprising:
responsive to the user declining to take the hearing test, applying a default profile.

3. The computer-implemented method of claim 1, wherein implementing the threshold-level testing includes:
instructing the auditory device to play a test sound at a listening band;
determining whether a confirmation was received that the user heard the test sound;
responsive to not receiving the confirmation, instructing the auditory device to increase a decibel level of the test sound until the confirmation is received or the test sound is played at a decibel level that meets a decibel threshold;
responsive to receiving the confirmation that the user heard the test sound or the test sound was played at the decibel threshold, advancing the listening band to a subsequent increment; and
continuing to repeat previous steps until the listening band meets a total listening band.

4. The computer-implemented method of claim 3, further comprising:
generating a user interface with an option for the user to select a number of listening bands.

5. The computer-implemented method of claim 3, wherein:
the threshold-level testing includes playing background noise with the test sound, and
the background noise is at least one selected from the group of white noise, voices, music, and combinations thereof.

6. The computer-implemented method of claim 1, wherein implementing the frequency gain balance testing includes:
instructing the auditory device to play a first test sound at listening band N and a second test sound at listening band N+1, wherein the first test sound is a reference test sound;
determining whether a confirmation was received that the first test sound and the second test sound were perceived to be a same volume;
responsive to not receiving the confirmation, raising a decibel level of the second test sound until the first test sound and the second test sound are perceived to be the same volume;
responsive to receiving the confirmation, advancing the listening band N so that N=N+1, wherein the second test sound becomes the reference test sound and is compared to a subsequent test sound at listening band N+1;
continuing to repeat the previous steps until the listening band N meets a total listening band; and
updating the hearing profile to include the frequency gain balance testing.

7. The computer-implemented method of claim 6, wherein the first test sound is played at a decibel level at which conversations are held and the second test sound is played at a threshold of hearing for a corresponding listening band as determined during the threshold-level testing.

8. The computer-implemented method of claim 6, wherein:
the frequency gain balance testing includes repeating the previous steps while playing background noise with the first test sound and the second test sound, and
the background noise is at least one selected from the group of white noise, voices, music, and combinations thereof.

9. The computer-implemented method of claim 1, wherein implementing the speech-clarity testing includes:
instructing the auditory device to play a speaking test;
determining whether a confirmation was received that the user is satisfied with the speaking test;
responsive to not receiving the confirmation that the user is satisfied with the speaking test, modifying the speaking test;
continuing to repeat the previous steps until the user is satisfied with the speaking test;
determining whether the user wants to repeat the speaking test with a voice of a different gender; and
responsive to completing the previous steps with the voice of a different gender or the user not wanting to repeat the speaking test with the voice of a different gender, updating the hearing profile.

10. The computer-implemented method of claim 9, wherein implementing the speech-clarity testing further includes playing the speaking test with one or more background noises until the one or more background noises are played.

11. The computer-implemented method of claim 1, wherein:
the threshold-level testing, the frequency gain balance testing, and the speech-clarity testing are implemented on a first ear and then on a second ear; and
the hearing profile includes different profiles for the first ear and the second ear.

12. The computer-implemented method of claim 1, wherein the auditory device is a hearing aid, earbuds, headphones, or a speaker device.

13. The computer-implemented method of claim 1, further comprising:
determining one or more presets that correspond to user preferences; and
transmitting the hearing profile and the one or more presets to the auditory device.

14. A device comprising:
one or more processors; and
logic encoded in one or more non-transitory media for execution by the one or more processors and when executed are operable to:
receive a signal from an auditory device;
determine whether a user selected to take a hearing test;
implement threshold-level testing;
implement frequency gain balance testing;
implement speech-clarity testing; and
generate a hearing profile based on one or more selected from the group of the threshold-level testing, the frequency gain balance testing, the speech-clarity testing, and combinations thereof.

15. The device of claim 14, wherein implementing the threshold-level testing includes:
instructing the auditory device to play a test sound at a listening band;
determining whether a confirmation was received that the user heard the test sound;
responsive to not receiving the confirmation, instructing the auditory device to increase a decibel level of the test sound until the confirmation is received or the test sound is played at a decibel level that meets a decibel threshold;
responsive to receiving the confirmation that the user heard the test sound or the test sound was played at the decibel threshold, advancing the listening band to a subsequent increment; and
continuing to repeat previous steps until the listening band meets a total listening band.

16. The device of claim 14, wherein implementing the frequency gain balance testing includes:
instructing the auditory device to play a first test sound at listening band N and a second test sound at listening band N+1, wherein the first test sound is a reference test sound;
determining whether a confirmation was received that the first test sound and the second test sound were perceived to be a same volume;
responsive to not receiving the confirmation, raising a decibel level of the second test sound until the first test sound and the second test sound are perceived to be the same volume;
responsive to receiving the confirmation, advancing the listening band N so that N=N+1, wherein the second test sound becomes the reference test sound and is compared to a subsequent test sound at listening band N+1;
continuing to repeat the previous steps until the listening band N meets a total listening band; and
updating the hearing profile to include the frequency gain balance testing.

17. Software encoded in one or more computer-readable media for execution by the one or more processors on a user device and when executed is operable to:
receive a signal from an auditory device;
determine whether a user selected to take a hearing test;
implement threshold-level testing;
implement frequency gain balance testing;
implementing speech-clarity testing; and
generate a hearing profile based on one or more selected from the group of the threshold-level testing, the frequency gain balance testing, the speech-clarity testing, and combinations thereof.

18. The software of claim 17, wherein implementing the threshold-level testing includes:
instructing the auditory device to play a test sound at a listening band;
determining whether a confirmation was received that the user heard the test sound;
responsive to not receiving the confirmation, instructing the auditory device to increase a decibel level of the test sound until the confirmation is received or the test sound is played at a decibel level that meets a decibel threshold;
responsive to receiving the confirmation that the user heard the test sound or the test sound was played at the decibel threshold, advancing the listening band to a subsequent increment; and
continuing to repeat previous steps until the listening band meets a total listening band.

19. The software of claim 17, wherein implementing the frequency gain balance testing includes:
instructing the auditory device to play a first test sound at listening band N and a second test sound at listening band N+1, wherein the first test sound is a reference test sound;
determining whether a confirmation was received that the first test sound and the second test sound were perceived to be a same volume;
responsive to not receiving the confirmation, raising a decibel level of the second test sound until the first test sound and the second test sound are perceived to be the same volume;
responsive to receiving the confirmation, advancing the listening band N so that N=N+1, wherein the second test sound becomes the reference test sound and is compared to a subsequent test sound at listening band N+1;
continuing to repeat the previous steps until the listening band N meets a total listening band; and
updating the hearing profile to include the frequency gain balance testing. [symbol] . The software of claim 17, wherein implementing the speech-clarity testing includes:
instructing the auditory device to play a speaking test;
determining whether a confirmation was received that the user is satisfied with the speaking test;
responsive to not receiving the confirmation that the user is satisfied with the speaking test, modifying the speaking test;
continuing to repeat the previous steps until the user is satisfied with the speaking test;
determining whether the user wants to repeat the speaking test with a voice of a different gender; and
responsive to completing the previous steps with the voice of a different gender or the user not wanting to repeat the speaking test with the voice of a different gender, updating the hearing profile.
